(19) United States
(12) Patent Application Publication
(10) Pub. No.: US 2014/0100500 A1
(43) Pub. Date: Apr. 10, 2014

(54) TORSO COMPRESSION MEDICAL DEVICE AND METHOD

(71) Applicant: AGEAS MEDICAL COMPRESSION, L.L.C., Wyoming, MI (US)

(72) Inventors: Scott M. Idsinga, Nunica, MI (US); Robb T. Idsinga, Nunica, MI (US); Daniel Dunn, Wyoming, MI (US)

(73) Assignee: AGEAS MEDICAL COMPRESSION, L.L.C., Wyoming, MI (US)

(21) Appl. No.: 13/741,778

(22) Filed: Jan. 15, 2013

Related U.S. Application Data
(60) Provisional application No. 61/795,043, filed on Oct. 5, 2012.

(51) Int. Cl. A61F 5/02

(52) U.S. Cl. CPC A61F 5/02
USPC 602/19

(57) ABSTRACT

A torso compression medical device allows selectively applied compressive torso support to alleviate chest pain. The device includes a flexible band of material having a width of at least several inches and a length sufficient to extend substantially around the person's torso, with handles on ends of the band positioned adjacent the person's hands. The handles can be grasped by the person and biased to selectively generate compression forces on the person's torso from wrapping tension on the band of material. Cushions on the band distribute forces from the tensioned band onto the person's torso. Related methods of use are disclosed, including wearing the device as a garment.

This utility application claims benefit under 35 USC section 119(e) of provisional application Ser. No. 61/795,043, filed Oct. 5, 2012, entitled TORSO COMPRESSION GARMENT, the entire contents of which are incorporated herein.

### BACKGROUND

The present invention relates to a medical apparatus used by a person with painful torso condition to selectively create chest compression forces to reduce pain and discomfort during movement.

Many people have painful chest conditions where any movement (such as deep breathing, coughing, rolling over, or standing up) causes severe chest pain. Examples include: a rib injury (including broken, bruised, or fractured ribs), respiratory issues, surgical patients including transplant or chest wound recovery, some muscular conditions, and many other circumstances. Often the pain of movement is so great that people may hold their chest, such as with folded arms or a pillow. However, this is not as effective as desired since it is limited to a front region of one's chest, and also the support is not as widespread and all-encompassing as desired. Historically, there is no real treatment for these conditions other than to minimize movement as much as possible and/or to use mild to narcotic painkillers. However, minimizing movement can cause shallow breathing, leading to pneumonia. Also, it is inconsistent with daily living. The use of painkillers is also not a preferred alternative due to side effects and masking of health issues.

Sometimes, a large elastic band is fastened tight around a person's chest to provide a constant chest compression force. See Hubbard U.S. Pat. No. 4,787,381. However, medical studies have shown that constant compressive torso pressure is not good for people, since it can lead to medical issues, such as restricting a person's ability to deep breath (potentially resulting in pneumonia). Also, constant torso pressure can adversely affect healing. Also, attachment of the elastic band is difficult, and cannot be done by the patient alone, which causes people to tend to leave it on for extended periods of time. This potentially leads to medical issues as noted above.

### SUMMARY OF THE PRESENT INVENTION

In one aspect of the present invention, a torso compression medical device is provided for a person having a painful chest condition where compressive torso support facilitates the person's movement. The device includes a band of material having a width of at least several inches and a length sufficient to extend substantially around the person's torso with ends of the band positioned generally adjacent the person's hands, handles secured to the ends that are configured and adapted to be grasped by the person and biased to generate compression forces on the person's torso from tension on the band of material, and at least one cushion on the band positioned to distribute forces from the band onto the person's torso.

In another aspect of the present invention, a method is provided for selectively creating torso compression forces, where the method includes providing a band with handles on each end sufficient in length to extend substantially around a person's torso and having at least one cushion on the band, placing the band around the person's torso with the handles adjacent the person's hands and the at least one cushion adjacent the torso, and selectively grasping and biasing the handles to generate tension in the band and thus creating compression forces on the person's torso, with the person controlling a biasing force and thus controlling compression forces that support the person's torso when moving.

An object of the present invention is to provide a device to allow people to better control pain, thus reducing the amount of pain medicine required, while providing a device people themselves can apply and selectively apply with as much or as little force as needed (and only when needed).

An object of the present invention is to provide a device to allow people to breathe more deeply while reducing the amount of pain medicine required.

An object of the present invention is to provide a non-drug mechanical device that is simple to use and that can be easily mechanically applied.

An object of the present invention is to provide a device allowing self-control of pain, thus resulting in greater movement and deeper breathing in persons, thus allowing people to obtain benefits such as reduced incidence of pneumonia and other problems associated with lack of physical activity and shallow breathing, allowing people greater independence during daily living.

An object of the present device is to provide an ability to significantly reduce pain in a hospital and/or home environment, enabling people to consume lesser amounts of painkillers (or eliminate them altogether), which in many cases are narcotic drugs. Notably, narcotic drugs, by their nature, reduce the natural breathing ability in a person and especially restrict deep breathing, creating a scenario where pneumonia is more likely to set in. The present device greatly reduces the possibility of that happening because the person not only avoids that impediment, but is able to breathe naturally to the very best of their ability, given their circumstances.

An object of the present invention is to help people with various chest pain issues (described above) be more comfortable with their injury and/or disease, speed the healing process while reducing their pain, yet without (or with less) use of drugs, and while continuing to give them a greatest degree of control and independence.

An object of the present invention is to provide a device that can be used by people having chest pain conditions including at least any of the following medical conditions: broken ribs, fractured ribs, costochondritis, osteoporosis (a disease where bones lose density making them more susceptible to bone fracture), osteoarthritis, osteopenia, rheumatoid arthritis, pneumonia, heart transplant, afflictions involving excessive coughing, pleurisy, tuberculosis, whooping cough, chest pain from heart disease and/or angina, shingles, lymphoma, myeloma, and certain other types of cancer. The present device is useful from injuries occurring from direct impact (such as motor vehicle accidents, falls, child abuse, contact sports), repetitive trauma (sports such as golf or rowing, severe and prolonged coughing spells), where there are significant risk factors (such as osteoporosis, sports participation, cancerous lesions in a rib making it more susceptible to breaks), and where complications are causing pain (such as a punctured lung, a lacerated spleen, liver, or kidney, pneumonia). The above items listed are not “stretch applications”, but instead illustrate the types of people who could benefit directly and greatly from use of the present device.

The present device provides an effective solution to all kinds of chest pains as described above through selectively generated compressive torso support where forces are self-generated by the “patient.” Movement is the most direct cause of pain from broken, fractured, or extremely sore ribs, or from a respiratory issue. In short, reduced or “supported” movement directly and immediately reduces the person's pain. An object of the present device is to reduce unwanted unsupported movement during an activity, be it breathing, coughing, rolling, or other movement. Our testing shows that by wrapping the present device around oneself and pulling on (or pressing on) the handles, pain can be reduced anywhere from 20 percent to 100 percent, depending on the condition and circumstances. Notably, the present device can be worn much like a garment, thus making it available at any moment. Further, a person wearing the device can adjust the amount of chest compression forces by adjusting a direction and/or amount of tension on the device's handles, with maximum support provided at the moment needed, thus adjusting pain control “on the fly”, making whatever adjustment is needed in pressure when coughing or moving. This avoids problems associated with a constant chest compressive force, such as shallow breathing (lack of deep breathing).

Using the present device, I believe that a wearer can breathe normally about 95+percent of the time (or more), varying from that only when they are placing tension on the device. Further, when using the device, they will be either moving about or taking deeper breathes than they would be able to without it. Notably, most severe chest wall injuries have historically been treated non-operatively, thus making the present device useful even in severe cases. In some recent studies, surgeons have shown interest in severe cases in rib fracture fixation surgery. However, the present device potentially allows a person to avoid such a surgery altogether, which itself can potentially be a tremendous advantage.

These and other aspects, objects, and features of the present invention will be understood and appreciated by those skilled in the art upon studying the following specification, claims, and appended drawings.

### BRIEF DESCRIPTION OF DRAWINGS

FIGS. 1-3 are front, side, and back views of the present medical device wrapped around a user, including a cushioned band with loop handles grasped by the user and biased forwardly for creating torso compression.

FIG. 4 is a front view similar to FIG. 1, but with the cushioned band wrapped tight around the user's torso including across a front of the user.

FIG. 5 is a top view of FIG. 1, and FIGS. 6-8 are horizontal cross sectional views through FIG. 5 and showing biasing directions for the device's handles to generate the forces VI-VIII in FIG. 5.

FIGS. 9-10 are front and side views of the device of FIG. 1 (laid out flat).

FIGS. 11-12 are cross sectional views along lines XI-XI and XII-XII in FIGS. 9-10.

FIG. 13 is a side view of an alternative cushion comprising an air bladder.

FIG. 14 is a front view of an alternative device that includes hook-and-loop-attached adjustable handles and double-loop handles.

FIGS. 15 is a fragmentary side view of a device including at least two cushion-receiving pockets and closed with a snap closure, and FIG. 16 is an enlarged fragmentary side view of a snap closure from FIG. 15.

FIG. 17 is a fragmentary front view of an alternative handle with reinforced attachment, and

FIG. 18 is a cross section taken along line XVIII in FIG. 17.

FIG. 19 is an exploded perspective view of an alternative device where components are all attached together by hook-and-loop material, thus facilitating removal, cleaning, and replacement/substitution of individual components.

### DETAILED DESCRIPTION OF PREFERRED EMBODIMENTS

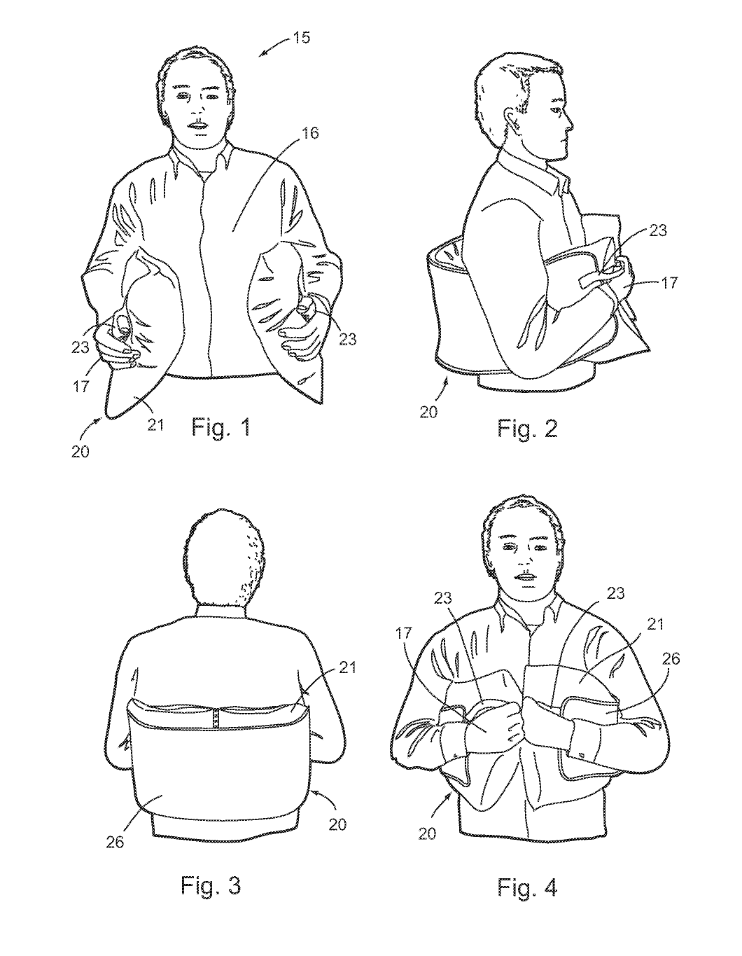

A torso compression medical device 20 (FIGS. 1-8) is provided for a person 15 having a painful chest condition in their torso 16. By gripping the device, the person 15 can selectively generate compressive torso support to reduce pain (such as when deep breathing or coughing) and/or to facilitate the person's movement (such as when rolling on a bed, or moving onto or from a chair). The device 20 includes a non-stretchable flexible band of material 21 sewn to enclose pillow-like cushions 24 (four being illustrated), potentially reinforced by a “backboard” support 26, and includes handles 23 at each end in a location for grasping by the person. The device 20 can have a width and length as necessary for a particular person's preference, whether adult or child, and obese or non-obese or underweight.

For example, I have a typical physical build for an adult person of about 160 pounds weight and 5′10″ height. My device 20 has a width of several inches (such as about 14″) and a length (such as about 45″ or longer). The device 20 is sufficiently long to extend substantially around my torso 16 with ends of the band positioned generally adjacent my hands 17. The handles 23 are loops secured to the ends 22 where they can be easily grasped. By pressing on (or pulling on or otherwise biasing) the handles 23, I can generate an optimal amount of compression forces on my torso from tension on the band of material 21 against the cushions 24. By varying the direction, amount, and timing of force on the handles 23, I can provide a level of control for the amount of torso support that I require, based on my personal need (e.g. coughing, breathing, moving, etc.).

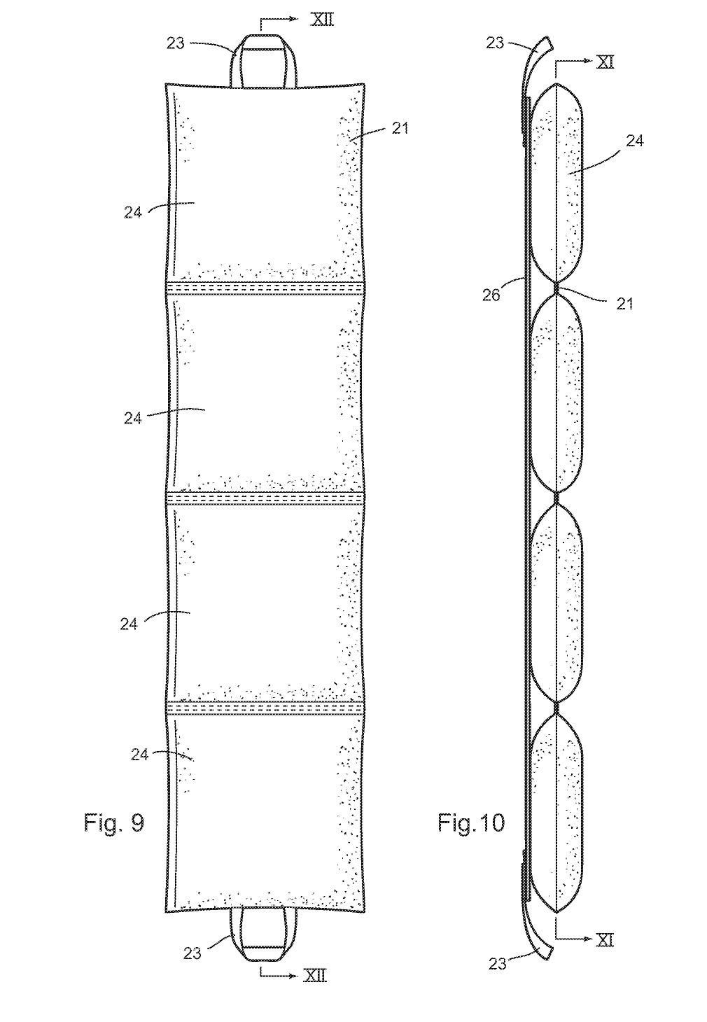

As noted above, the band 21 (FIGS. 9-12) can vary in length as needed, such as from about 30″ to 55″ long (preferably about 45″-55″ for most adult people) and from about 12″ to 16″ wide (preferably about 14″ for most adult people). Dimensions can be varied as necessary for particular body shapes and personal preferences. Notably, the device can be sized for children, mid-sized adults, large adults, or obese adults. The illustrated band 21 is sewn to include multiple pockets 25 (four rectangular pockets being illustrated). The pockets 25 in the illustrated band of material 21, are sewn closed. However, it is contemplated that each pocket 25 can be open on one side (such as on a top side) for receiving a pillow-like cushion 24 (also called a “pillow” herein). In such case, the pockets 25 would include a releasable closure, such as a snap or hook-and-loop material, (or snap 27, FIG. 16) as noted below. The illustrated pockets 25 (and cushions 24) are all the same size, but it is contemplated that the pockets can be different sizes, and/or can be a continuous single pocket extending around the person. Similarly, the cushions can also be different sizes and thicknesses as needed for a particular patient. Further it is contemplated that larger or smaller cushions 24 can be substituted for optimal use or patient preference.

As noted above, the illustrated device 20 (FIGS. 9-12) includes a plurality of cushions 24 (four pillow-shaped cushions are illustrated) which are permanently sewn into and carried in pockets 25 on the band 21. The cushions 24 are positioned inside the band 21 in a location where they comfortably distribute forces from the band 21 onto the person's torso 16. The pockets 25 and cushions 24 are designed with mating shapes to maintain a desired position of the cushions 24 during use of the device (i.e. so that the cushions do not migrate to one end of the pockets during use). The band 21 itself can be non-stretchable durable fabric, such as is commercially available. Alternatively, it is contemplated that the band can be flexible or semi-stiff sheet of fabric or plastic, reinforced and supported as needed, and/or it can include a semi-rigid (flexible) “backboard” support 26 (also called a “backboard” herein) attached to the band 21, with the “backboard” support 26 providing a more uniform distribution of stress from the handles across the device 20 from top to bottom, and around the device 20. By using the “backboard” support 26, the band 21 itself can potentially be made of a lower cost, more comfortable, lighter-weight material. Alternatively, the band 21 can be reinforced with strong cords (not specifically shown) that extend longitudinally from handle 23 to handle 23 (much like a hammock), with cords being positioned along edges of the band 21 and near the handles 23 where forces are greatest.

Where the pockets 25 are open on one side, they can be releasably closed by a pillow retainer or closure device, such as a snap(s) 27 (FIGS. 15-16). The snaps 27 positively retain the cushions 24 therein, but allow the cushions 24 to be removed, cleaned, reassembled, and/or replaced. It is contemplated that the retainer/closure device (27) could be any fastener, such as Velcro® hook-and-loop material, a hook, a tie, a loop and button, or other retainer means. Also, it is contemplated that the cushions 24 could be held in place by a fastener inside the pocket 25, such as hook-and-loop material located inside the mating pocket 25. It is also contemplated that the cushions 24 could be attached to an inside surface of the belt 21 or support 26, eliminating the pockets (25) altogether.

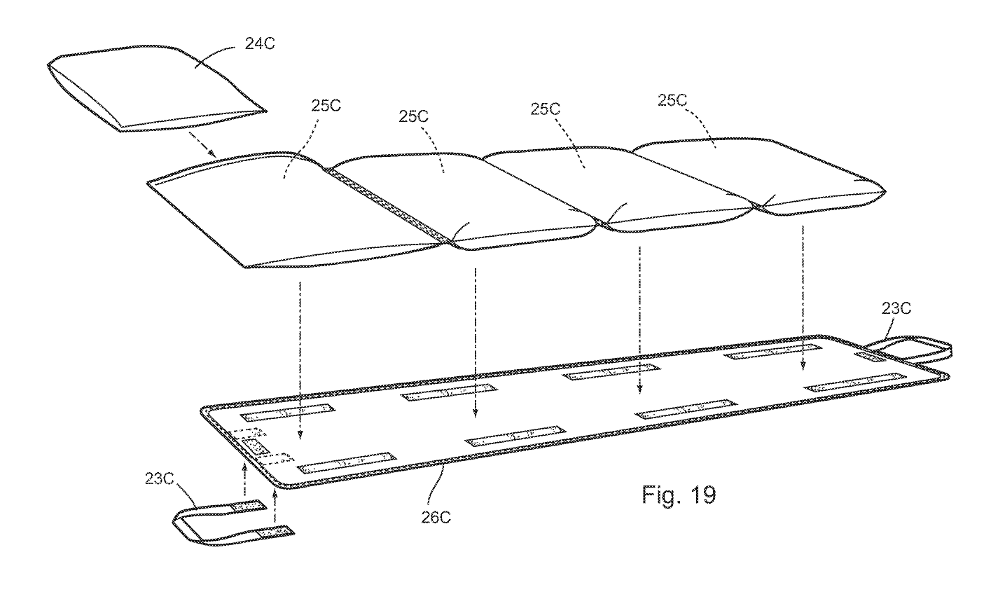

The illustrated pillow-like cushions 24 are 14″×14″ pillows made of cloth and containing polystyrene fiber, foam rubber, or other cushioning material. However, it is contemplated that many different materials could be used, including potentially an air-containing inflatable bladder 24A that can be adjusted in size and thickness on-site (see FIG. 13). Specifically, the bladder 24A would include a short tube and close-off valve 24B, allowing a person to blow air into the bladder and lock it closed, or alternatively it could include on the short tube and hand pump bulb (not specifically shown) that can be squeezed to fill the bladder. It is contemplated that the entire device 20 can be made cleanable, washable, and/or otherwise sterilizable. Alternatively, it is contemplated that individual components can be made removable and/or cleanable, washable, and/or otherwise sterilizable (such as via removable cushions 24 as in FIGS. 15-16, or via complete disassembly as in FIG. 19).

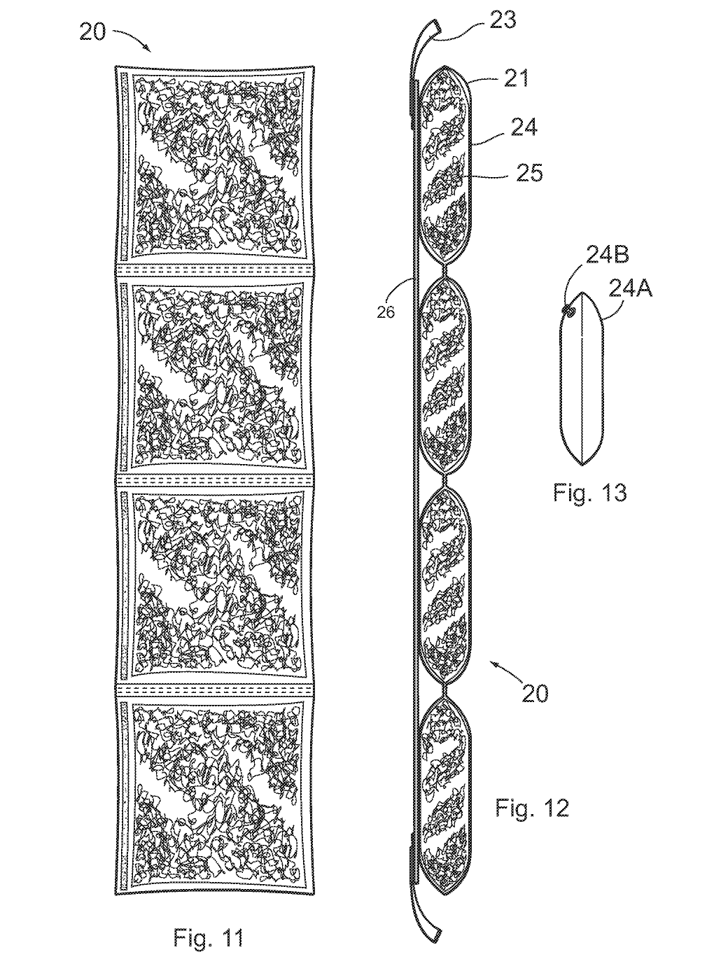

Notably, the illustrated cushion 24 (FIGS. 11-12) is essentially a cloth-covered pillow having an internal cushion material covered with an outer covering of fabric. The illustrated cushion 24 is 14″×14″, and is relatively square with pointed corners and bellowed center, which makes its corners somewhat pointed and its edges somewhat concave shaped. The pointed corners and concave edge shape help keep the cushion 24 in a square and centered position in the associated pocket 25 in the device 20 during use. As noted above, this “positive” location can be assisted by including a retainer, such as hook-and-loop (Velcro®) material inside the pockets 25.

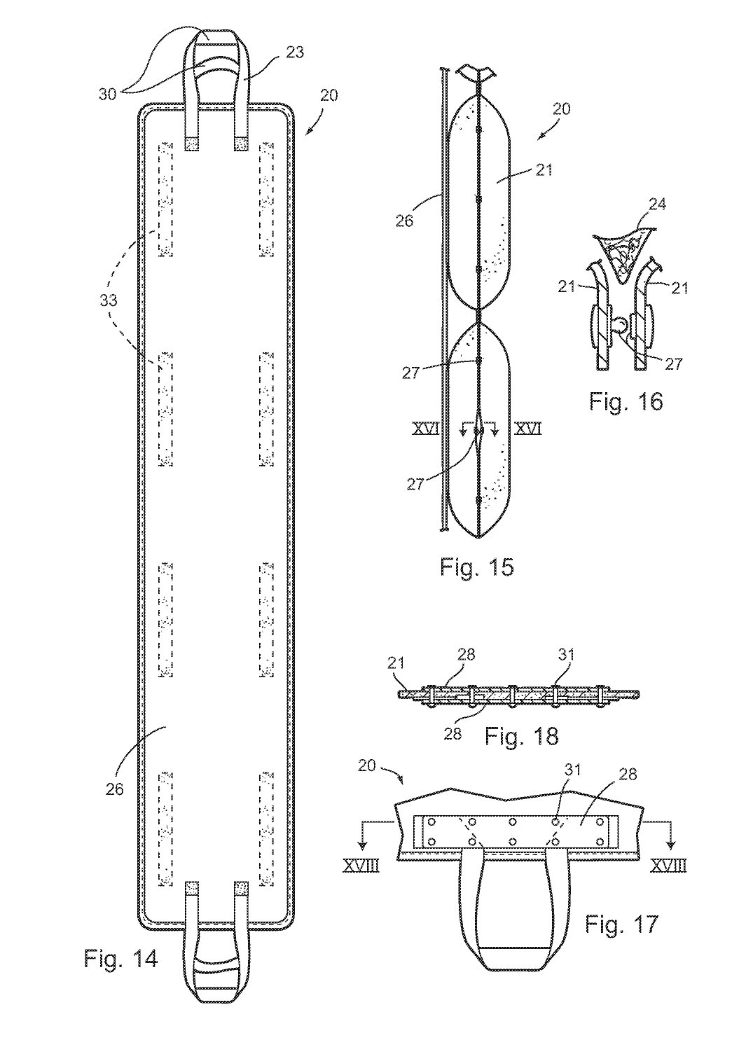

The two illustrated handles 23 (FIGS. 9-10) are each a loop of fabric, cloth or plastic, and are attached to one of the ends 22 by stitching sufficient to endure the stress of use. Specifically, the handles 23 are sufficiently strong and comfortably shaped so that a person can apply as much force as desired to tension the device 20 which in turn provides compressive forces around the person's torso 15 (see FIGS. 1-8). The illustrated handles 23 (FIG. 12) are secured by double stitched seams 27. It is contemplated that the handles 23 can be attached by whatever means necessary for strength and durability, such as by double or triple stitching the seams or by sewing a strip of reinforcing plastic 28 (FIG. 18) into the connection. It also is contemplated that the handles 23 can be longer or shorter as desired, or can include a multiple grips 30 (see double-grip handles 23 in FIG. 14). Alternatively, it is contemplated that the handles 23 can be adjustably secured with hook-and-loop material (FIG. 19), such that they can be released and reconnected at different lengths, thus making the handle adjustable in length. Also, the handles 23 can be attached by different means, such as by fastening screws 31 or rivets (FIG. 18) that extend through the overlapping components. Friction strips 33 (FIG. 14) can be used to help keep the device 20 in place on a person when desired.

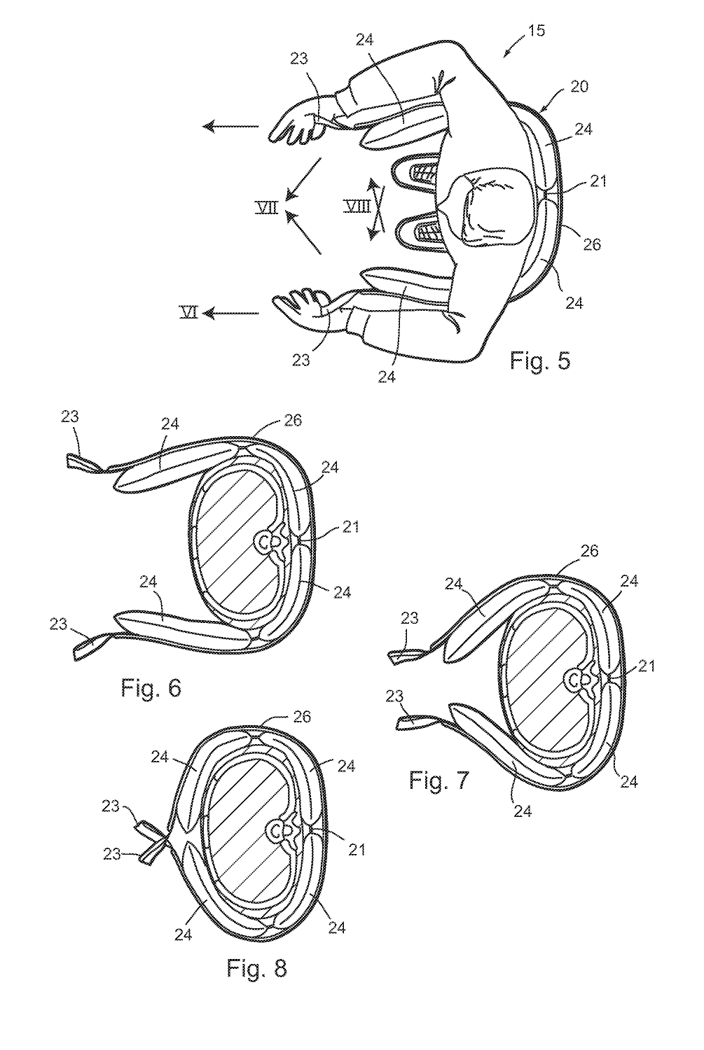

The device 20 (FIGS. 1-4) creates a torso belt that can be worn around the human torso 16 like a garment. When thus positioned, it can be used/tensioned by the person as needed. Specifically, when desired, the person can apply as much tension as desired, in any direction desired, and at an optimal time in relation to the movement (i.e. applied simultaneously with the rise and fall of a cough, or with the extended support needed when rolling over, or with the twist and full-around-the-torso support needed when rising from a chair (see FIGS. 5-8). Specifically, FIG. 5 illustrates three different ways/methods that a person can grip and bias the handles 23 to create a compressive force by the band 21 around the person's torso. In FIG. 6, the RH and LH forces 31 extend in a forward direction, and are generated by the person biasing/pressing forward on the handles 23. In FIG. 7, the RH and LH forces 32 extend in an angled forward direction, and are generated by the person biasing/pressing forward and toward a center of the person, which provides greater side support to the person's torso, such as during a cough. In FIG. 8, the RH and LH forces 33 extend around the person including laterally across the person's front, and are generated by the person gripping opposing handles 23 and pulling across one's body, thus creating an inward compressive force that extends substantially completely around the person's torso in a manner providing significant inward compressive force in all areas, including in a center/front of the person's torso.

FIG. 19 illustrates a modified device 20C where all components are releasably secured together, such as by using hook-and-loop material. In particular, in device 20C, the backboard support 26C includes hook-and-loop patches for releasably secure attachment of the handles 23C and for secure attachment of the pockets 25C/band 21C. The illustrated pockets 25C include an open side into which a cushion 24C fits, with the open side being held closed by hook-and-loop strips (not specifically shown) along the open side. It is contemplated that the backboard support 26C can be used or not used in a given customized device. Notably, it is contemplated that the pockets 25C can be made of a single sheet of material folded over and sewn with seams to form individual pockets 25C, or alternatively can be made of separate sheets, each folded to make a single pocket 25C, with the series of pockets 25C being held together by hook-and-loop strips extending across overlapping ends of the pockets 25C. By this later arrangement, a particular device (20C) can be assembled as a custom medical device specifically designed to meet particular patient's needs and preferences, including making the device 20C as long or short as needed, with more (or fewer) cushions, more (or fewer) pockets, with thicker (and/or thinner) cushions in selected locations, and with larger (or smaller handles). Also, the device 20C can be broken down for compact shipment. Also, individual components can be rolled up (such as the pockets and handles) or compressed (such as the cushions) for dense shipment. Also, the device 20C can be completely broken down for cleaning, sanitizing, replacement of worn components, and/or for using components on another custom built torso compression device.

It is to be understood that variations and modifications can be made on the aforementioned structure without departing from the concepts of the present invention, and further it is to be understood that such concepts are intended to be covered by the following claims unless these claims by their language expressly state otherwise.

The embodiments of the invention in which an exclusive property or privilege is claimed are defined as follows:

1. A torso compression medical device for a person having a painful chest condition where compressive torso support facilitates the person's movement, comprising:
a band of material having a width of at least several inches and a length sufficient to extend partially around the person's torso with ends of the band positioned generally adjacent the person's hands; and
handles secured to the ends that are configured and adapted to be grasped by the person and biased to generate compression forces on the person's torso from tension on the band of material.

2. The medical device of claim 1, including at least one cushion on the band and positioned to distribute forces from the band onto the person's torso.

3. The medical device of claim 2, wherein the band is a one-piece fabric sewn to form pockets that receive the at least one cushion.

4. The medical device of claim 1, wherein the band is a semi-rigid flexible band perforated with a pattern to provide flexibility in the band.

5. The medical device of claim 1, wherein the band includes edges that are rounded to reduce abrasion or cutting of the person's skin.

6. The medical device of claim 1, wherein the band includes hook-and-loop fastener material and at least one cushion attached to the hook-and-loop fastener material.

7. The medical device of claim 1, wherein the band includes a first layer of fabric material and a second layer of semi-rigid support material attached to the fabric material in at least two locations.

8. A torso compression medical device for a person having a painful chest condition where compressive torso support facilitates the person's movement, comprising:
an elongated support with multiple pillows shaped to extend partially around the person's torso; and
handles secured to outer ends of the elongated support that are configured, positioned, and adapted to be grasped by the person and biased to generate compression forces on the person's torso from tension on the elongated support and pillows.

9. A method of creating torso compression forces for a person having a painful chest condition where compressive torso support facilitates the person's movement, comprising:
providing a band with handles on each end sufficient in length to extend partially around a person's torso;
placing the band around the person's torso with the handles adjacent the person's hands; and
grasping and biasing the handles to selectively generate tension in the band and thus creating compression forces on the person's torso, with the person controlling a biasing force and thus controlling compression forces that support the person's torso when moving.

10. The method defined in claim 9, including providing at least one cushion on the band, and wherein placing the band includes placing the at least one cushion inside the band adjacent the person's torso.
